(19) United States
(12) Patent Application Publication
(10) Pub. No.: US 2015/0259315 A1
(43) Pub. Date: Sep. 17, 2015

(54) GRAPE EXTRACT, DIETARY SUPPLEMENT THEREOF, AND PROCESSES THEREFOR

(71) Applicant: Constellation Brands, Inc., Victor, NY (US)

(72) Inventor: Anil Jageshwar SHRIKHANDE, Madera, CA (US)

(73) Assignee: Constellation Brands, Inc., Victor, NY (US)

(21) Appl. No.: 14/212,837

(22) Filed: Mar. 14, 2014

(51) Int. Cl. C07D 311/76; A23L 2/52; A23L 1/30

(52) U.S. Cl. CPC C07D 311/76; A23L 1/3002; A23L 2/52; A23V 2002/00

(57) ABSTRACT

The present invention relates to a novel grape extract, particularly from white grape seeds, and processes for making such, that is useful for, inter alia, modulating oxidative stress, inflammation, and impaired insulin sensitivity in an individual, particularly an individual suffering from Metabolic Syndrome. The processes of the present invention produce a highly concentrated polyphenol product by maximizing extraction of monomeric and oligomeric procyanidins and minimizing extraction of polymeric procyanidins. The grape extract of the present invention comprises between about 5-15% monomers, about 5-20% dimers, about 3-10% trimers, about 2-10% tetramers, and about 2-10% pentamers by weight with a high percentage of galloylation. The grape extract of the present invention can be formulated into dietary supplements or pharmaceutical compositions, including capsules, tablets, powders, solutions, gels, suspensions, creams, gels, and the like. These dietary supplements in, for instance, powder or solution form, may be added to nutraceuticals, foods and/or beverages to form functional nutraceutical, food, and/or beverage products.

### FIELD OF THE INVENTION

This invention relates to a novel grape extract and processes for producing such grape extract. This novel grape extract is useful for, inter alia, modulating oxidative stress, inflammation, and impaired insulin sensitivity in an individual, particularly an individual suffering from Metabolic Syndrome. This invention also relates to a dietary supplement comprising the grape extract of the present invention.

### BACKGROUND

Grape seeds contain about 5-8% by weight flavonoids. Flavonoids constitute an important group of dietary polyphenolic compounds that are widely distributed in plants. More than 4000 chemically unique flavonoids have been identified in plant sources, such as fruits, vegetables, legumes, nuts, seeds, herbs, spices, flowers, as well as in beverages such as tea, cocoa, beer, wine, and grape juice.

The terminology of flavonoids with respect to grape seeds refers to monomeric flavan-3-ols, specifically (+)-catechin, (−)-epicatechin, and (−)-epicatechin 3-gallate. Two or more flavan-3-ol monomers chemically linked are called proanthocyanidins or oligomeric proanthocyanidins (“OPCs”), which includes procyanidins and prodelphinidins. OPCs containing two monomers are called dimers, three monomers are called trimers, four monomers are called tetramers, five monomers are called pentamers, etc. Operationally, the oligomers have chain lengths of 2 to 7 (dimers to heptamers); whereas polymers represent components with chain lengths greater than 7. After considerable discussion, it was the consensus of the Grape Seed Method Evaluation Committee (through the National Nutritional Foods Association) to define OPCs as all proanthocyanidins containing two or more monomers, including polymers or condensed tannins. Thus, oligomers in grape extracts include, for instance, dimers and trimers, and there is evidence that the polymers can have as many as sixteen units.

Below is a typical structure of a proanthocyanidin, showing epicatechin-gallate extension units and terminal units. The extension units are represented, for instance, by the epicatechin (2) and epigallocatechin (3) linking groups. Whereas, a terminal unit is represented by the epicatechin gallate (4) group.

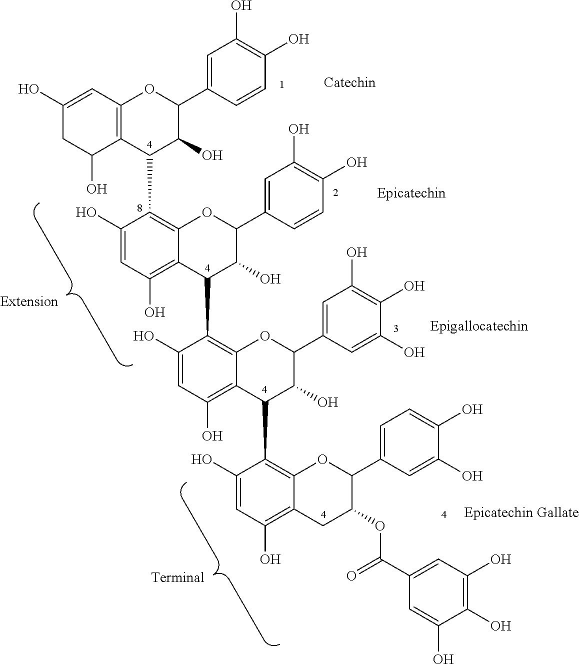

In order for polyphenolic compounds to be used commercially as a grape extract, these compounds have to be separated from grapes in a more concentrated form. The general process in which the polyphenolic compounds are extracted, purified and concentrated from whole grapes, grape pomace and grape seeds is disclosed in U.S. Pat. No. 6,544,581, which is incorporated herein by reference.

In addition to antioxidant activities, flavonoids have been reported, in animal studies, to exert anti-cancer effects by reducing growth of new blood vessels, and to have anti-inflammatory, anti-microbial, and anti-allergenic activities. It has also been found that the grape extract of the present invention may be used to modulate oxidative stress, inflammation, and impaired insulin sensitivity in an individual, particularly for an individual suffering from Metabolic Syndrome. Metabolic syndrome (Met.S) is a growing medical problem in industrialized countries and is diagnosed when three of the following factors are present: abdominal obesity, elevated serum triglycerides, low serum high density lipoprotein (HDL) concentration, elevated blood pressure, and elevated blood glucose. The syndrome is associated with insulin-resistance, impaired glucose control, atherogenic dyslipidemia, oxidative stress, and enhanced cardiovascular risk.

“Metabolic Syndrome,” also called “Syndrome X,” the “Insulin Resistance Syndrome,” or the “Deadly Quartet,” is characterized by an accumulation of risk factors for cardiovascular disease, stroke and/or diabetes mellitus type II. Metabolic Syndrome may be caused by an overproduction of cortisol, a stress hormone, which causes an accumulation of fat inside the abdominal cavity and insulin resistance. Drug therapy is not currently recommended for individuals with Metabolic Syndrome. The risk factors that characterize Metabolic Syndrome include an increased amount of adipose tissue inside the abdominal cavity (abdominal obesity), insulin resistance with increased risk of developing diabetes, hyperinsulinemia, high levels of blood fats, increased blood pressure, and elevated serum lipids. The National Cholesterol Education Adult Treatment Panel (ATP III) defined Metabolic Syndrome as individuals having at least three of the following risk factors:

| Risk Factor | Defining Level |
|---|---|
| Abdominal obesity, given as waist | |
| circumference*^{†} | |
| Men | >102 cm (>40 in) |
| Women | >88 cm (>35 in) |
| Triglycerides | ≧150 mg/dL |
| HDL cholesterol | |
| Men | <40 mg/Dl |
| Women | <50 mg/dL |
| Blood pressure | ≧130/≧85 mm Hg |
| Fasting glucose | ≧110 mg/dL^{‡} |

*Overweight and obesity are associated with insulin resistance and Metabolic Syndrome. The presence of abdominal obesity, however, is more highly correlated with the metabolic risk factors than is an elevated BMI. Therefore, the simple measure of waist circumference is recommended to identify the body weight component of Metabolic Syndrome.
^{†}Some male patients can develop multiple metabolic risk factors when the waist circumference is only marginally increased, e.g., 94 to 102 cm (37 to 39 in). Such patients may have a strong genetic contribution to insulin resistance. They should benefit from changes in life habits, similarly to men with categorical increases in waist circumference.
^{‡}The American Diabetes Association has recently established a cut-off point of ≧100 mg/dL, above which individuals have either pre-diabetes (impaired fasting glucose) or diabetes. This new cut-off point should be applicable for identifying the lower boundary to define an elevated glucose as one criterion for Metabolic Syndrome.

Conditions related to Metabolic Syndrome include diabetes mellitus type II, dyslipoproteinemia, myocardial infarction, stroke and other arteriosclerotic diseases, as well as the risk factors for these diseases, including insulin resistance in general, abdominal obesity caused by accumulation of intra-abdominal fat, elevated blood serum lipids and glucose, raised diastolic and/or systolic blood pressure, and hypertension.

Insulin resistance, a characteristic feature of metabolic syndrome (MetS), is known to be associated with impaired glucose tolerance and impaired fasting glucose. Consumption of a meal that is high in readily available carbohydrates and fat causes postprandial increases in glycemia and lipidemia and markers of oxidative stress, inflammation and insulin resistance.

Hyperglycemia has been shown to impose cellular oxidative stress through the increased generation of endogenous reactive oxygen species (ROS) particularly in adipose tissue, both in vitro and in vivo. Increased ROS, in turn promotes inflammation by activating nuclear factor-kappaB (NF-κB) transcription complex resulting in the expression of a number of genes coding for pro-inflammatory cytokines.

Oxidative stress and inflammation are believed to play a critical role in the pathogenesis of several diseases including atherosclerosis and diabetes. Oxidative stress impairs glucose uptake in muscle and fat and decreases insulin secretion from β cells of the pancreas, resulting in prolonged hyperglycemia, increased glycation endproducts and endothelial dysfunction, all of which contribute to the development of atherosclerosis. Since insulin resistance presents before the development of diabetes, early intervention strategies designed to reduce insulin resistance and improve glucose control could ameliorate unfavorable effects on blood vessels and risk for micro- and macro-vascular disease. One possible approach for decreasing insulin resistance and improving glucose control is to enhance the anti-oxidant status of the body.

As described previously consumption of a meal that is high in readily available carbohydrates and fat results in a postprandial increase in markers of oxidative stress, inflammation and insulin resistance (See, Burton-Freeman et al. I, (Burton-Freeman B, Talbot J, Park E, Krishnankutty S, Edirisinghe I., Mol Nutr Food Res. 2012 Feb. 14. doi: 10.1002/mnfr.201100649. [Epub ahead of print] PubMed PMID: 22331646); Burton-Freeman et al. II, (Burton-Freeman B, Linares A, Hyson D, Kappagoda T., J Am Coll Nutr. 2010 February; 29(1):46-54); and Edirisinghe et al., (Edirisinghe I, Banaszewski K, Cappozzo J, Sandhya K, Ellis C L, Tadapaneni R, Kappagoda C T, Burton-Freeman B M., Br J Nutr. 2011 September; 106(6):913-22).). These changes are accentuated in states where insulin function is impaired such as in MetS (REF). It has been suggested that fruits and vegetables, particularly those with a higher polyphenolic content, have favorable effects on human health due to their ability to modulate oxidative and inflammatory stress in peripheral tissues (See, Rahman I., Nutr Rev. 2008 August; 66 Suppl 1:S42-5; and Rahman I, Biswas S K, Kirkham P A, Biochem Pharmacol. 2006 Nov. 30; 72(11):1439-52.). Grape seeds are a concentrated source of polyphenols and have received considerable attention for their antioxidant capacity and biological effects (See, Leifert W R, Abeywardena M Y, Nutr Res 2008; 28:729-37; Chis I C, Ungureanu M I, Marton A, Simedrea R, Muresan A, Postescu I D, Decea N., Diab Vasc Dis Res. 2009 July; 6(3):200-4; Meeprom A, Sompong W, Suwannaphet W, Yibchok-anun S, Adisakwattana S., Br J Nutr. 2011 October; 106(8):1173-81; and Kim Y, Choi Y, Ham H, Jeong H S, Lee J., J Med Food. 2012 Mar. 8. [Epub ahead of print] PubMed PMID: 22400909.).

Further, pre-hypertensive individuals are classified as individuals that have systolic pressure between 120 and 139 mmHg or have diastolic pressure between 81 and 89 mmHG. This classification is based on the Seventh Report of the Joint National Committee on Prevention, Detection, Evaluation, and Treatment of High Blood Pressure (JNC 7), page 87, NIH Publication No. 04-5230. Pre-hypertensive individuals are not typically treated with drug therapy, but rather are given suggestions for a healthy lifestyle. These suggestions include maintaining a healthy weight; being physically active; following a healthy eating plan that emphasizes fruits, vegetables, and low fat dairy foods; choosing and preparing foods with less sodium; and drinking alcoholic beverages in moderation if at all. Adopting healthy lifestyle habits is usually an effective first step in both preventing and controlling abnormal blood pressure.

There is a need for a grape extract and a dietary supplement comprising such grape extract that can be used as adjunctive therapy, which is effective for providing health benefits such as modulating the oxidative stress, inflammation, and impaired insulin sensitivity in patients with metabolic syndrome (Met.S).

### SUMMARY OF THE INVENTION

In one embodiment, the present invention provides a grape seed extract comprising about 5-35% by weight epicatechin-gallate terminal units. The grape extract of the present invention may also comprise about 5-40% by weight epicatechin-gallate extension units.

In another embodiment, the present invention provides a method for preparing a grape extract comprising; step (1) heating grape seeds, dry or fresh, with hot water for a time sufficient to extract most of the polyphenols; step (2) separating the crude grape seed-water extract from spent seeds by draining over metal screens and cooling the crude grape seed-water extract, and optionally treating the cooled crude grape seed-water extract with a pectolytic enzyme; and step (3) acidifying the resulting grape seed extract with an acid, preferably a mineral acid, more preferably with sulfuric acid, to a pH of approximately 1.5-2.5 and which is allowed to react from about one hour to about two days, wherein the grape seeds are from white grapes, preferably chardonnay grapes.

In yet another embodiment, the present invention provides a pharmaceutical composition comprising a grape seed extract and a pharmaceutical acceptable excipient for modulating post-prandial oxidative stress, inflammation, impaired insulin sensitivity, or a combination thereof in a subject suffering from Metabolic Syndrome.

In another embodiment there is provided for a use of a grape seed extract for the preparation of a medicament for treating or preventing Metabolic Syndrome or treating or preventing type II diabetes in subject in a pre-diabetic condition and suffering from Metabolic Syndrome.

### DETAILED DESCRIPTION

The present invention provides a grape extract that is effective in modulating the oxidative stress, inflammation, and impaired insulin sensitivity in patients with metabolic syndrome (Met.S). In general, the grape extract of the present invention comprises about 5-15% monomers, about 5-20% dimers, about 3-10% trimers, about 2-10% tetramers, and about 2-10% pentamers by weight. The total amount of low molecular weight phenolic compounds including monomers, dimers, trimers, tetramers, and pentamers is between about 25-50% by weight, preferably between about 25-40% by weight, more preferably between about 30-40% by weight, and more preferably between about 25-35% by weight. The total amount of phenolic compounds is about 80% by weight or more, and preferably about 90% by weight or more.

In one embodiment, the grape extract of the present invention comprises about 6-15% monomers, about 7-15% monomers, about 8-15% monomers, about 9-15% monomers, about 10-15% monomers, about 11-15% monomers, about 12-15% monomers, about 13-15% monomer, and about 14-15% monomers. In another embodiment, the grape extract of the present invention comprises about 5-14% monomers, about 5-13% monomers, about 5-12% monomers, about 5-11% monomers, about 5-10% monomers, about 5-9% monomers, about 5-8% monomers, about 5-7% monomer, and about 5-6% monomers. In yet another embodiment, the amount of monomer in the present invention is selected from the group consisting of about 5%, 6%, 7%, 8%, 9%, 10%, 11%, 12%, 13%, 14%, and 15%.

In one embodiment, the grape extract of the present invention comprises about 6-20% dimers, about 7-20% dimers, about 8-20% dimers, about 9-20% dimers, about 10-20% dimers, about 11-20% dimers, about 12-20% dimers, about 13-20% dimers, about 14-20% dimers, about 15-20% dimers, about 16-20% dimers, about 17-20% dimers, about 18-20% dimers, and about 19-20% dimers. In another embodiment, the grape extract of the present invention comprises about 5-19% dimers, about 5-18% dimers, about 5-17% dimers, about 5-16% dimers, about 5-15% dimers, about 5-14% dimers, about 5-13% dimers, about 5-12% dimers, about 5-11% dimers, about 5-10% dimers, about 5-9% dimers, about 5-8% dimers, about 5-7% dimers, and about 5-6% dimers. In yet another embodiment, the amount of dimer in the present invention is selected from the group consisting of about 5%, 6%_{,} 7%_{,} 8%_{,} 9%_{,} 10%_{,} 11%_{,} 12%_{,} 13%, 14%, 15%, 16%, 17%, 18%, 19%, and 20%.

In one embodiment, the grape extract of the present invention comprises about 4-10% trimers, about 5-10% trimers, about 6-10% trimers, about 7-10% trimers, about 8-10% trimers, and about 9-10% trimers. In another embodiment, the grape extract of the present invention comprises about 3-9% trimers, about 3-8% trimers, about 3-7% trimers, about 3-6% trimers, about 3-5% trimers, and about 3-4% trimers. In yet another embodiment, the amount of trimer in the present invention is selected from the group consisting of about 3%, 4%_{,} 5%, 6%, 7%, 8%, 9%, and 10%.

In one embodiment, the grape extract of the present invention comprises about 3-10% tetramers, about 4-10% tetramers, about 5-10% tetramers, about 6-10% tetramers, about 7-10% tetramers, about 8-10% tetramers, and about 9-10% tetramers. In another embodiment, the grape extract of the present invention comprises about 2-9% tetramers, about 2-8% tetramers, about 2-7% tetramers, about 2-6% tetramers, about 2-5% tetramers, about 2-4% tetramers; and about 2-3% tetramers. In yet another embodiment, the amount of tetramer in the present invention is selected from the group consisting of about 2%, 3%, 4%, 5%, 6%, 7%, 8%, 9%, and 10%.

In one embodiment, the grape extract of the present invention comprises about 3-10% pentamers, about 4-10% pentamers, about 5-10% pentamers, about 6-10% pentamers, about 7-10% pentamers, about 8-10% pentamers, and about 9-10% pentamers. In another embodiment, the grape extract of the present invention comprises about 2-9% pentamers, about 2-8% pentamers, about 2-7% pentamers, about 2-6% pentamers, about 2-5% pentamers, about 2-4% pentamers; and about 2-3% pentamers. In yet another embodiment, the amount of pentamer in the present invention is selected from the group consisting of about 2%, 3%, 4%, 5%, 6%, 7%, 8%, 9%, and 10%.

In one embodiment, the total amount of low molecular weight phenolic compounds, i.e. monomers, dimers, trimers, tetramers, and pentamers, is about 25% or greater, about 30% or greater, about 35% or greater, about 40% or greater, about 45% or greater up to about 50% by weight. In another embodiment, the total amount of low molecular weight phenolic compounds is about 25% or greater, 26% or greater, 27% or greater, 28% or greater, 29% or greater, 30% or greater, 31% or greater, 32% or greater, 33% or greater, 34% or greater, 35% or greater, 36% or greater, 37% or greater, 38% or greater, 39% or greater, 40% or greater, 41% or greater, 42% or greater, 43% or greater, 44% or greater, 45% or greater, 46% or greater, 47% or greater, 48% or greater, 49% or greater up to about 50% by weight.

The grape extract of the present invention has a phenolic profile, as determined by normal-phase high-performance liquid chromatography (“HPLC”), of about 5-15% monomers, about 5-20% dimers, about 4-10% trimers, about 2-10% tetramers, and about 2-10% pentamers by weight. The grape extract of the present invention also comprises about 80% by weight or more total phenolic compounds, and preferably about 90% by weight or more, as determined by the Folin Ciocalteu method.

The grape extract of the present invention also comprises about 5-35% by weight epicatechin-gallate terminal units, more preferably about 8-25% by weight, even more preferably 10-20%, or comprises 5% by weight or more epicatechin gallate terminal units, preferably 8-9% by weight or more epicatechin gallate units, even more preferably the grape seed extract comprises 12% by weight or more of epicatechin gallate terminal units, as determined by reverse-phase HPLC after thiolysis reaction. The grape extract of the present invention also comprises about 5-40% by weight epicatechin-gallate extension units, preferably about 12-35% by weight, and more preferably about 15-25% by weight, as determined by reverse-phase HPLC after thiolysis reaction.

The grape extract of the present invention is produced by modifying the hot water extraction process disclosed in U.S. Pat. No. 6,544,581 as described below. In general, the hot water extraction process, as disclosed in the '581 patent, involves the following steps. In step (1), grape seeds, dry or fresh, may be heated with hot water for a time sufficient to extract most of the polyphenols. Temperatures of 140-212° F. may be employed, preferably 160-212° F., more preferably 180-212° F., yet more preferably 190-212° F., for a period of about 1-6 hours. The time of heating may be varied in relation to the temperature used. Generally, lower temperatures require longer extraction times. In step (2), the crude grape seed-water extract may be separated from spent seeds by draining over metal screens. The extract may then be cooled and optionally treated with any suitable commercially available pectolytic enzyme, such as Pectinex® Ultra SP-L manufactured by Novo Nordisk, at a concentration of about 50-200 ppm to break down cell wall constituents. Preferably, the seed water extract may be enzyme-treated for a period of two hours at a temperature of 80-120° F. Alternatively, the seed-water extract may be enzyme-treated for 7-14 days or longer at about 40-50° F. In step (3), the resulting turbid seed extract may be acidified with an acid, preferably a mineral acid, more preferably with sulfuric acid, to a pH of approximately 1.5-2.5 and allowed to react from about one hour to about two days. The acidified extract may be cooled for up to several weeks to allow for macromolecules, including proteins and other polysaccharides, to settle. The cooled acidified extract may then be filtered using diatomaceous earth to yield a clarified seed extract. Other filter aids, such as perlite, may also be used.

In the above process the grape seeds are specifically selected for the presence of proanthocyanidins with a high level of galloylation. Suitable grape seeds are those from white grapes, preferably from Chardonnay grapes alone or in combination with other varieties such Saugnion Blanc, Muscat, French Colambard, Chenin Blanc, Pinot Grigio.

The grape extract of the present invention may be formulated into dietary supplements or pharmaceutical compositions, including capsules, tablets, powders, solutions, gels, suspensions, creams, pastes, gels, suppositories, transdermal patches, and the like. These dietary supplements in, for instance, powder or solution form, may be added to nutraceuticals, foods and/or beverages to form functional nutraceutical, food, and/or beverage products. The dietary supplements may be formulated as powders, for example, for mixing with consumable liquids such as milk, juice, water or consumable gels or syrups for mixing into other dietary liquids or foods. The dietary supplements of this invention may be formulated with other foods or liquids to provide pre-measured supplemental foods, such as single serving bars. Typical food products that may incorporate the grape extract of the present invention include dairy foods such as yogurt, cereals, breads, snack food products, fruit juices and other soft drinks. Flavorings, binders, protein, complex carbohydrates, vitamins, minerals and the like may be added as needed. Preferably, the grape extract is formulated for oral administration.

The present invention also provides a dietary supplement or pharmaceutical composition comprising the grape extract of the invention. The dietary supplement of pharmaceutical composition, when administered to a mammal, including humans, modulatest post-prandial oxidative stress, inflammation, impaired insulin sensitivity, or a combination thereof in a subject suffering from Metabolic Syndrome.

The dietary supplements or pharmaceutical compositions of the present invention are intended for daily administration or as needed. The magnitude of a prophylactic or therapeutic dose of the dietary supplement or pharmaceutical composition in individuals will vary with the severity of the condition being treated and the route of administration. The dose, and perhaps the dose frequency, will also vary according to the age, body weight, and response of the individual. In general, the total daily dose range, for the conditions described herein, is from about 50 mg to about 1,000 mg grape extract administered in single or divided doses orally, topically, or transdermally, preferably orally. A preferred oral daily dose range is from about 50 mg to about 500 mg of the grape extract (i.e., excluding excipients and carriers), more preferably about 150 mg to about 300 mg. For example, capsules or tablets may be formulated in either 150 mg or 300 mg doses, whereas beverages can be formulated with 50 mg of grape extract of the present invention. Such a regimen of administration is preferably maintained for at least one month, more preferably six months or longer.

The dietary supplements or pharmaceutical compositions of the present invention may be formulated in a conventional manner (i.e. by dry mixing, dry or wet granulation, direct compression), in admixture with pharmaceutically acceptable carriers, excipients, vitamins, minerals and/or other nutrients. Representative carriers and excipients include, but are not limited to, starches, sugars, microcrystalline cellulose, diluents, granulating agents, lubricants, binders, disintegrating agents, and the like, in the case of oral solid preparations (such as powders, capsules, and tablets).

Any suitable route of administration may be employed to administer the dietary supplements or pharmaceutical compositions of the invention to an individual. Suitable routes include, for example, oral, rectal, parenteral, intravenous, topical, transdermal, subcutaneous, and intramuscular. Although any suitable route of administration may be employed for providing the patient with an effective amount of the grape extract according to the methods of the present invention, oral administration is preferred, including solid dosage forms such as tablets, capsules, or powders. It is also preferred that the grape extract is formulated for use in functional nutraceutical, food, or beverage products.

The grape extract of the present invention can also be combined with other active agents including but not limited to diuretics, beta-blockers, ACE inhibitors, angiotensin antagonists, calcium channel blockers, alpha-blockers, alpha-beta-blockers, nervous system inhibitors, vasodilators, antioxidants.

Pharmaceutical formulations of the present invention contain the grape seed extract as described herein. In addition to the active ingredient(s), the pharmaceutical formulations of the present invention may contain one or more excipients. Excipients are added to the formulation for a variety of purposes.

Diluents may be added to the formulations of a present invention. Diluents increase the bulk of a solid pharmaceutical composition, and may make a pharmaceutical dosage for containing the composition easier for the patient and caregiver to handle. Diluents for solid compositions include, for example, microcrystalline cellulose (e.g., AVICEL®, microfine cellulose, lactose, starch, pregelatinized starch, calcium carbonate, calcium sulfate, sugar, dextrates, dextrin, dextrose, dibasic calcium phosphate, dehydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatine, guar gum, hydrogenated vegetable oil, hydroxyethyl cellulose, hydroxypropyl cellulose (e.g., KLUCEL®), hydroxypropyl methyl cellulose (e.g., METHOCEL®), liquid glucose, magnesium aluminium silicate, maltodextrin, methylcellulose, polymethacrylates, povidone (e.g., KOLLIDON® PALSDONE®), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL®, PRIMELOSE®), colloidal silicon dioxide, croscarmellose sodium, crospovidone (e.g., KOLLIDON®, POLYPLASDONE®), guar gum, magnesium aluminium silicate, methyl cellulose, microcrystalline cellulose, polacrilin potassium, powdered cellulose, pregelatinized starch, sodium alginate, sodium starch glycolate (e.g., EXPLOTAB®), and starch.

Glidants can be added to improve the flowability of a non-compacted solid composition, and to improve the accuracy of dosing. Excipients that may function as glidants include colloidal silicon dioxide, magnesium trisilicate, powdered cellulose, starch, talc, and tribasic calcium phosphate.

When a dosage form such as tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion, and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavour enhancers make the dosage form more palatable to the patient. Common flavoring agents and flavour enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance, and/or facilitate patient identification of the product and unit dosage level.

In liquid pharmaceutical compositions prepared using grape seed extract, the grape seed extract and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatine guar gum, hydroxyethyl cellulose, hydroxypropyl cellulose, hydroxpropyl methyl cellulose, maltodextrin, polyvinyl alcohol, povidone, propylene carbonate, propylene glycol alginate, sodium alginate, sodium starch glycolate, starch tragacanth, and xantham gum.

Sweetening agents such as sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated, hydroxyanisole, and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant, and ophthalmic, administration. Although the most suitable administration in any given case will depend on the nature and severity of the condition being treated, the most preferred route of the present invention is oral.

Dosage forms include solid dosage forms like tablets, powders, capsules, suppositories, sachets, troches, and lozenges, as well as liquid syrups, suspensions, elixirs, and in beverages.

The dosage form of the present invention may be a capsule containing the composition, preferably a powdered or granulated solid composition of the invention, within either a hard or soft shell. The shell may be made from gelatin, and, optionally, contain a plasticizer such as glycerine and sorbitol, and an opacifying agent or colorant.

A composition for tableting or capsule filling may be prepared by wet granulation. In wet granulation, some or all of the active ingredients and excipients in powder form are blended, and then further mixed in the presence of a liquid, typically water, that causes the powders to clump into granules. The granulate is screened and/or milled, dried, and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet, and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules.

Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The active ingredient and excipients may be formulated into compositions and dosage forms according to methods know in the art.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

1. Characterization of Grape Extracts

### Reverse-Phase HPLC Procedure to Determine Percent of Monomers, Oligomers, and Polymers

Reverse-phase HPLC analysis of grape extract can be used to determine the proportion of monomers, oligomers and polymers based on peak area at 280 nm.

HPLC Conditions:
Mobile Phase: A: 2% glacial acetic acid
   B: 80% acetonitrile, 0.4% acetic acid
Gradient:

| Time (min) | % A | % B | Curve |
|---|---|---|---|
| 0.00 | 100 | 0 | — |
| 3.00 | 100 | 0 | 6 |
| 6.00 | 96 | 4 | 6 |
| 15.00 | 90 | 10 | 6 |
| 30.00 | 85 | 15 | 6 |
| 50.00 | 77 | 23 | 6 |
| 60.00 | 75 | 25 | 6 |
| 66.00 | 70 | 30 | 6 |
| 80.00 | 50 | 50 | 6 |
| 83.00 | 20 | 80 | 6 |
| 85.00 | 100 | 0 | 6 |
| 105.00 | 100 | 0 | 6 |
| 110.00 | 100 | 0 | 6 |

Column: 250 mm×4.6 mm, Prodigy 5μ, ODS (3) 100 Å (Phenomenex, Torrance, Calif.)
Flow rate: 1.0 mL/min
Detection wavelength: 280 nm
Temperature: 30° C.
Injection: 25 μL

Sample Preparation:

Accurately weigh 0.1 g grape extract into a 100 mL volumetric flask. Dissolve the sample in a small amount of methanol (≦5 mL), sonicate if necessary. Fill to volume with 18 Megaohm water. Centrifuge the sample (14,000 rpm, 10 min) or filter through 0.45 μM glass filter prior to injection. Determination for percent by weight monomers, oligomers and polymers is based on the peak area and concentration of the standards.

Method to Determine Terminal and Extensional Units of Proanthocyanidins Based on HPLC Analysis after Thiolysis Reaction

Thiolysis is a method to determine average molecular size (degree of polymerization) and basic structure of proanthocyanidins in grape extract. The information provided may indicate biological quality of grape extract for nutritional absorption in the body.

Thiolysis Reagent:

5% phenyl methanethiol (benzyl mercaptan) in methanol containing 0.2 N HCl.

Condition:

0.1% Grape extract methanol solution was mixed with an equal volume of thiolysis reagent, stirred, and heated at 90° C. for 2 min. Water was added to stop the reaction. The reactant was then centrifuged at 14000 rpm for 2 min. The supernatant was analyzed directly by HPLC.

HPLC Conditions:
Mobile Phase: A: 10% acetic acid/0.1% TFA/5% acetonitrile/84.9% water (v/v/v/v)
   B: acetonitrile
Gradient:

| | |
|---|---|
| 0-30 min | 0-50% B |
| 30-35 min | 50-100% B |

Column: 150 cm×2.0 mm i.d., 4 μm Synergi hydro-RP 80 Å (Phenomenex, Torrance, Calif.)
Flow rate: 0.3 mL/min
Detection wavelength: HP 1100 FLD with excitation @ 276 nm and emission @ 316 nm and HP DAD at 280 nm
Temperature: 30° C.
Injection: 1-3 μL

The grape extracts to be analyzed were dissolved in methanol, mixed with an equal volume of thiolytic reagent and heated for 2 min at 90° C. The released units were identified by mass spectrometry and quantitatively determined by HPLC under the conditions above. The average degree of polymerization was measured by calculating the molar ratio of all flavan-3-ol units (thioether adducts plus terminal units) to catechin, epicatechin and epicatechin-gallate corresponding to terminal units. The percentage of epicatechin gallate terminal units were determined based on molar ratio of epicatechin gallate in the sum of total moles of terminal units, which includes catechin, epicatechin and epicatechin gallate. The percentage of epicatechin-gallate extension units were determined based on molar ratio of epicatechin gallate thioether adducts in the sum of total moles of thioether adducts of extension units, which include catechin, epicatechin and epicatechin-gallate thioether adducts. The total amount of phenolic compounds was quantified in terms of grams Gallic Acid Equivalents (GAE) by the Folin Ciocalteu method. For more details on the Folin Ciocalteu analysis procedure, see: Waterhouse, A. L., Determination of Total Phenolics, in Current Protocols in Food Analytical Chemistry, I1.1.1-I1.1.8, Wrolstad, R. E., Wiley, 2001, or Singleton, V. L.; Orthofer, R.; Lamuela-Raventos, R. M. “Analysis of total phenols and other oxidation substrates and antioxidants by means of Folin-Ciocalteu Reagent,” Methods in Enzymology 1999, 299, 152-178, both of which are incorporated herein by reference.

### Normal-Phase HPLC Analysis for Proanthocyanidins

HPLC Analysis of Proanthocyanidins:

Chromatographic analyses were performed on an HP 1100 series HPLC equipped with an autosample/injector, binary pump, column heater, diode array detector, fluorescence detector, and HP ChemStation for data collection and manipulation. Normal phase separations of proanthocyanidin oligomers were performed on a Phenomenex Luna Silica (2) column.
Mobile Phase: A: dichloromethane, methanol, water, and acetic acid (83:13:2:2 (v/v))
   B: methanol, water, and acetic acid (96:2:2 (v/v))
Gradient:

| | |
|---|---|
| 0-30 min | linear 0-17.6% B |
| 30-45 min | linear 17.6-30.7% B |
| 45-50 min | linear 30.7-87.8% B |
| 50-60 min | linear 87.8% B |

Column: Phenomenex LUNA Silica (3.0×150 mm; 3.0 μm)
Flow rate: 0.5 mL/min
Detection: HP 1100 FLD with excitation @ 276 nm and emission @ 316 nm
Temperature: 25° C.
Injection: 3 μL

In all cases, the column was re-equilibrated between injections with equivalent of 5 mL of the initial mobile phase. Catechin standards were prepared and analyzed to establish a response calibration curve from which to calculate the concentration of proanthocyanidins in the samples. Relative response factors of dimers, trimers, tetramers and pentamers to monomers with fluorescence detection were reported by R. L. Prior and L. Gu, “Occurrence and biological significance of proanthocyanidins in American diet,” Phytochemistry 2005, 66(18) 2264-2280, using standards isolated and purified from cocoa bean. These response factors were used to calculate dimers, trimers, tetramers and pentamers relative to monomers.

### EXAMPLES

The invention is further defined by reference to the following examples describing a process for making the grape extract and preparing the dietary supplements. The examples are representative, and they should not be construed to limit the scope of the invention.

### Example 1

### Process for Making the Grape Extract

Dried grape seeds from Chardonnay grapes were extracted with water at a temperature of 200° F. for two hours and the extract was separated from the seeds on metal screens. The extract was cooled to 90-100° F. and pectinase was added at a concentration of 200 ppm. The resulting extracts were acidified to a pH of 1.5 to 2.5, which allowed flocculation of proteins and polysaccharides on cooler storage from 40-60° F. The extract was filtered and processed further according to the '581 patent to produce a grape extract with characteristics modulating post-prandial oxidative stress, inflammation, impaired insulin sensitivity, or a combination thereof in a subject suffering from Metabolic Syndrome.

### Example 2

### Grape Seed Extract

The grape seed extract prepared has a total phenolic level of 94.3 gallic acid equivalents (wt/wt) as assessed by the Folin & Ciocalteu method. The moisture content is 4.1%. The detailed polyphenolic composition as assessed using LC-MS/MS is given in Table 1.

TABLE 1
Polyphenolic composition of the Grape Seed Extract (GSE) *

| Name of the compound | % composition |
|---|---|
| Total Gallic Acid | 5.7 |
| Catechin | 6.7 |
| Epicatechin | 4.8 |
| Dimer B | 34.4 |
| Polymer | 46.1 |

### Example 3

### Capsule

Grape extract of the invention (150 mg or 300 mg) is dry mixed with magnesium stearate (3 mg or 6 mg respectively) and loaded into hard shell gelatin capsules (made of gelatin and water). In the 150 mg formulation, the grape extract has a minimum of 90% phenols or 135 mg of phenols per 150 mg of grape extract. In the 300 mg formulation, the grape extract has a minimum of 90% phenols or 270 mg of phenols per 300 mg of grape extract. The daily dosage is one capsule per day.

### Example 4

### Powder

Grape extract of the invention is formulated into a dry mix with the excipients as shown in Table 2 to be used in a beverage, wherein the ingredients are dry blended. To prepare the final beverage, 9.47 g of the dry mix is combined with 500 mL of cold water and stirred. A 500 mL serving contains 16 calories. The final beverage contains 100 mg grape extract and 120 mg vitamin C per 1 L serving, which will have an ORAC value of 2200 TE.

ORAC, measured in mmoles Trolox (a noncommercial, water-soluble derivative of tocopherol) equivalents (TE) per gram, stands for “Oxygen Radical Absorbance Capacity.” This is the standard by which scientists measure antioxidant activity in foods and supplements. A single servings of fresh or freshly cooked fruits and vegetables supply an average of 600 to 800 ORAC units. It has been suggested that increasing intake of foods or supplements that provide 2,000 to 5,000 ORAC units per day may have health benefits.

TABLE 2

| Ingredients | % Dry Mix (g) |
|---|---|
| Maltodextrin | 37.48 |
| Citric Acid | 29.99 |
| Clouding Agent (Purity Gum 2000)* | 5.25 |
| Aspartame | 3.85 |
| Sodium Citrate, FCC Grade | 3.75 |
| Ultra Guar** | 3.75 |
| N&A Orange Flavor (SN313897)*** | 7.5 |
| Nat FF Passion Fruit Flavor (SN 313898)*** | 4.27 |
| FD&C Yellow #6 (20:1 in Maltodextrin) | 2.24 |
| FD&C Yellow #5 (20:1 in Maltodextrin) | 0.75 |
| Ascorbic Acid | 0.64 |
| Grape Extract | 0.53 |
| TOTAL | 100 |

*Available from National Starch & Chemical Corporation, Bridgewater, NJ
**Available From P.L. Thomas & Co., Inc. Morristown, NJ
***Available from International Flavors & Fragrances, Dayton, NJ

### Example 5

### Vitamin/Mineral Supplement

Grape seed extract of the invention (150 mg) is dry mixed with the following excipients listed in Table 3 and pressed into a tablet to form a multi-vitamin/mineral supplement. The daily dosage is one tablet per day, preferably taken with food.

TABLE 3

| Ingredients | % Daily Value |
|---|---|
| Vitamin A 3500 IU (29% as Beta Carotene) | 70 |
| Vitamin C 60 mg | 100 |
| Vitamin D 400 IU | 100 |
| Vitamin E 45 IU | 150 |
| Vitamin K 10 mcg | 13 |
| Thiamin 1.5 mg | 100 |
| Riboflavin 1.7 mg | 100 |
| Niacin 20 mg | 100 |
| Vitamin B6 3 mg | 150 |
| Folic Acid 400 mcg | 100 |
| Vitamin B12 25 mcg | 417 |
| Biotin 30 mcg | 10 |
| Pantothenic Acid 10 mg | 100 |
| Calcium 299 mg | 20 |
| Phosphorus 48 mg | 5 |
| Iodine 150 mcg | 100 |
| Magnesium 100 mg | 25 |
| Zinc 15 mg | 100 |
| Selenium 20 mg | 29 |
| Copper 2 mg | 100 |
| Manganese 2 mg | 100 |
| Chromium 150 mcg | 125 |
| Molydenum 75 mcg | 100 |
| Chloride 72 mg | 2 |
| Potassium 80 mg | 2 |
| Grape Extract 150 mg | * |
| Boron 150 mcg | * |
| Nickel 5 mcg | * |
| Silicon 2 mg | * |
| Vanadium 10 mcg | * |
| Lutein 250 mcg | * |
| Lycopene 300 mcg | * |

* Daily Value (% DV) not established

### Example 6

### Vitamin/Mineral Supplement

Grape seed extract of the invention (150 mg) is blended with the following ingredients and excipients listed in Table 4 in V blender until uniform. The blend was pressed into tablets that reach a specified weight of 775 mg±2% to form a multi-vitamin/mineral supplement. The tablets is spray coated with a clear coating of a water soluble gum such as hydroxypropyl methylcellulose and dried. The daily dosage is one tablet per day. The batch size for the formulation in Table 3 is 500,000 Tablets.

TABLE 4

| | Label | | Overage | Amount/ | Amount/ |
|---|---|---|---|---|---|
| Ingredients (Units of Measure) | Claim | | (%)* | Tablet (mg) | Batch (Kg) |
| Vitamin A Palmitate @ 500K IU/gm (IU) | 5000 | IU | 30 | 13.000 | 6.500 |
| Vitamin D₃ @ 850K IU/g (IU) | 400 | IU | 30 | 0.612 | 0.306 |
| Vitamin E succinate (D-α) @ 1210 IU/g (IU) | 15 | IU | 5 | 13.017 | 6.508 |
| Vitamin C (mg) | 30 | mg | 2 | 30.600 | 15.300 |
| Thiamine HCl @ 89.2% (mg) | 1.5 | mg | 2 | 1.715 | 0.858 |
| Riboflavin (mg) | 1.7 | mg | 2 | 1.734 | 0.867 |
| Niacinamide (mg) | 10 | mg | 2 | 10.200 | 5.100 |
| Pyridoxine HCl 82.3% (mg) | 2 | mg | 5 | 2.552 | 1.276 |
| Folic Acid Trituration 1.0% (mcg) | 400 | mcg | 25 | 50.000 | 25.000 |
| Vitamin B-12 Trituration 1.0% (mcg) | 6 | mcg | 20 | 0.720 | 0.360 |
| Pantothenic Acid (Cal Pan.) (mg) | 10 | mg | 5 | 10.500 | 5.250 |
| Biotin Trituration 1.0% (mcg) | 30 | mcg | 20 | 3.600 | 1.800 |
| Calcium (Dicalcium Phosphate) 29.46% (mg) | 100 | mg | 0 | 344.119 | 172.060 |
| Phosphorus (Dicalcium Phosphate) 22.77% (mg) | 75 | mg | 0 | 0.000 | 0.000 |
| Magnesium (MgO) 60.32% (mg) | 20 | mg | 0 | 33.156 | 16.578 |
| Zinc (ZnO) 80.34 (mg) | 5 | mg | 0 | 6.224 | 3.112 |
| Iodine (KI) 76.45% (mcg) | 150 | mcg | 0 | 0.196 | 0.098 |
| Copper (Gluconate) 14.00% (mg) | 2 | mg | 0 | 14.286 | 7.143 |
| Manganese (Gluconate) 12.34% (mg) | 2 | mg | 0 | 16.207 | 8.104 |
| Grape Extract | 150 | mg | | 150.000 | 25.000 |
| Microcrystalline cellulose | | | | 33.750 | 16.875 |
| Croscarmellose Sodium | | | | 20.250 | 10.125 |
| Stearic Acid | | | | 13.500 | 6.750 |
| Magnesium Stearate | | | | 5.063 | 2.531 |
| TOTAL | | | | 775.000 | 337.500 |

*Percent amount of ingredient over label claim used to reach the label claim amount.

1. A process for making a polyphenol extract from grapes comprising the steps of: (1) heating a member selected from the group consisting of whole grapes, grape seeds, grape pomace, and mixtures thereof with water at a temperature of about 140°-212° F. to obtain a crude grape-water extract; and (2) cooling the crude grape-water extract; and step (3) acidifying the resulting grape seed extract with an acid to a pH of approximately 1.5-2.5, wherein the grape seeds are from white grapes.

2. The process of claim 1, wherein the grape seeds are from Chardonnay grapes alone or in combination with other varieties of white grape selected from Saugnion Blanc, Muscat, Pinot Grigio, and French Colamberd grapes.

3. The process of claim 1, wherein the cooled crude grape water extract is treated with a pectolytic enzyme at a temperature of about 80-120° F.

4. The process of claim 1 in which the contacting step (2) further comprises the steps of: (2a) separating the crude grape-water extract from insoluble grape solids and (2b) cooling the separated crude grape-water extract.

5. The process of claim 1 further comprising the steps of: (4) cooling the acidified polyphenol extract; and (5) filtering the cooled acidified polyphenol extract to obtain a filtered polyphenol extract.

6. The process of claim 5 in which the filtering step further comprises the step of: treating the cooled acidified polyphenol extract with an adsorbent resin to obtain a purified polyphenol extract.

7. A purified polyphenol extract produced according to the process of any of claims 1-6 wherein the extract comprises about 5-35% by weight epicatechin-gallate terminal units and about 5-40% by weight epicatechin-gallate extension units.

8. A polyphenol extract from grapes comprising between about 5-15% monomers, about 5-20% dimers, about 3-10% trimers, about 2-10% tetramers, about 2-10% pentamers by weight, and about 5-35% by weight of epicatechin-gallate terminal units.

9. The extract of claim 8, wherein the total amount of monomers, dimers, trimers, tetramers and pentamers is between about 25-50% by weight.

10. The extract of claim 8, comprising about 80% by weight or more total phenolic compounds.

11. The extract of claim 8, comprising about 5-40% by weight epicatechin-gallate extension units.

12. The extract of claim 8, comprising about 90% by weight or more total phenolic compounds.

13. The extract of claim 8, comprising about 10-25% by weight epicatechin-gallate terminal units.

14. The extract of claim 8, wherein the total amount of monomers, dimers, trimers, tetramers and pentamers is between about 25-50% by weight; the total amount of phenolic compounds is about 80% by weight or more; the total amount of epicatechin-gallate terminal units is about 5-35% by weight; and the total amount of epicatechin-gallate extension units is about 5-40% by weight.

15. A formulation for oral administration comprising the polyphenol extract of claim 8.

16. A food product comprising the polyphenol extract of claim 8.

17. A beverage comprising the polyphenol extract of claim 8.

18. A dietary supplement comprising the polyphenol extract of claim 8.

19. A nutraceutical product comprising the polyphenol extract of claim 8.

20. A pharmaceutical composition comprising the polyphenol extract of claim 8.

21. The pharmaceutical composition of claim 20 comprising a polyphenol extract from grapes having between about 5-15% monomers, about 5-20% dimers, about 3-10% trimers, about 2-10% tetramers, about 2-10% pentamers, and about 5-35% by weight epicatechin gallate terminal units and at least one pharmaceutically acceptable excipient.

22. The pharmaceutical composition of claim 21, wherein the polyphenol extract comprises about 80% by weight or more total phenolic compounds.

23. The pharmaceutical composition of claim 21, wherein the polyphenol extract comprises about 5-40% by weight epicatechin-gallate extension units.

24. The pharmaceutical composition of claim 21, wherein the polyphenol extract comprises about 90% by weight or more total phenolic compounds.

25. The pharmaceutical composition of claim 21, wherein the polyphenol extract comprises about 10-25% by weight epicatechin-gallate terminal units.
